(19) United States
(12) Patent Application Publication
(10) Pub. No.: US 2025/0221737 A1
(43) Pub. Date: Jul. 10, 2025

(54) BENDABLE OPTICAL TROCAR ASSEMBLY

(71) Applicant: MEDTRONIC, INC., Minneapolis, MN (US)

(72) Inventors: Vladimir P. Nikolski, Blaine, MN (US); Linnea R. Lentz, Stacy, MN (US); Kevin R. Seifert, Forest Lake, MN (US); Kyle R. Marquard, Lino Lakes, MN (US); Matthew J. Hoffman, Saint Paul, MN (US)

(21) Appl. No.: 18/855,942

(22) PCT Filed: Mar. 31, 2023
(86) PCT No.: PCT/IB2023/053258

§ 371 Date: Oct. 10, 2024

Related U.S. Application Data
(60) Provisional application No. 63/330,072, filed on Apr. 12, 2022.

(51) Int. Cl. A61B 17/34; A61B 1/313; A61B 17/00

(52) U.S. Cl. CPC A61B 17/3417; A61B 1/313; A61B 2017/00367; A61B 2017/00455

(57) ABSTRACT

The technology disclosed herein relates to, in part, an assembly having a handle and a tunneling shaft coupled to the handle. The tunneling shaft extends from the handle to a distal end, where a portion of the tunneling shaft extends in a curved orientation between the handle and the distal end. An optical window is disposed at the distal end of the tunneling shaft. An insertion pathway extends through the handle and the tunneling shaft to the optical window. A constriction mechanism is coupled to the handle and defines a portion of the insertion pathway. The constriction mechanism is configured to selectively constrict the insertion pathway. An endoscope is configured to be inserted in the handle to the optical window along the insertion pathway.

### TECHNOLOGICAL FIELD

The present disclosure is generally related to an optical trocar. More particularly, the present disclosure is related to a bendable optical trocar.

### SUMMARY

The technology disclosed herein generally relates to an assembly having a handle and a tunneling shaft coupled to the handle. The tunneling shaft extends from the handle to a distal end, where at least a portion of the tunneling shaft extends in a curved orientation between the handle and the distal end. An optical window is disposed at the distal end of the tunneling shaft. An insertion pathway extends through the handle and the tunneling shaft to the optical window. A constriction mechanism is coupled to the handle and defines a portion of the insertion pathway. The constriction mechanism is configured to selectively constrict the insertion pathway. An endoscope is configured to be inserted in the handle to the optical window along the insertion pathway.

In some such embodiments, the constriction mechanism has an inner diaphragm defining the portion of the insertion pathway. Additionally or alternatively, the constriction mechanism has a manually engageable knob. Additionally or alternatively, the constriction mechanism is configured for selective disengagement and reengagement of the endoscope. Additionally or alternatively, the assembly has an optical material disposed in the insertion pathway, where the optical material is in contact with the optical window. Additionally or alternatively, the assembly has an orientation indicator on the optical window. Additionally or alternatively, the constriction mechanism has a pressure limiter that is configured to limit constriction of the insertion pathway. Additionally or alternatively, the magnitude of constriction of the constriction mechanism is adjustable by a user. Additionally or alternatively, the tunneling shaft is bendable.

Some embodiments of the current technology relate to a method. An endoscope is inserted through an insertion pathway of a trocar assembly from a proximal end to an optical window disposed on a distal end of the trocar assembly. A constriction mechanism is engaged that is positioned towards the proximal end of the trocar assembly to secure the endoscope relative to the insertion pathway. A tunneling shaft of the trocar assembly is bent. The constriction mechanism is disengaged. The endoscope is repositioned within the insertion pathway. The constriction mechanism is re-engaged to secure the endoscope relative to the insertion pathway.

In some such embodiments, the constriction mechanism is engaged by pivoting a lever. Additionally or alternatively, the constriction mechanism is engaged by turning a knob. Additionally or alternatively, optical material is disposed within the insertion pathway. Additionally or alternatively, inserting the endoscope includes translating the endoscope through an optical material disposed in the insertion pathway. Additionally or alternatively, the constriction mechanism is configured to limit pressure on the endoscope. Additionally or alternatively, the optical window has an orientation indicator. Additionally or alternatively, the magnitude of the constriction is adjusted. Additionally or alternatively, adjusting the magnitude of the constriction includes adjusting an inner diaphragm. Additionally or alternatively, the distal end is inserted through an incision in a patient. Additionally or alternatively, cutting blades coupled to the distal end are deployed.

The above summary is not intended to describe each embodiment or every implementation. Rather, a more complete understanding of illustrative embodiments will become apparent and appreciated by reference to the following Detailed Description of Exemplary Embodiments and claims in view of the accompanying figures of the drawing.

### BRIEF DESCRIPTION OF THE DRAWINGS

The present technology may be more completely understood and appreciated in consideration of the following detailed description of various embodiments in connection with the accompanying drawings.

FIG. 1 is a perspective view of an example optical trocar consistent with embodiments.

FIG. 2 is a cross-sectional view of an example optical trocar consistent with FIG. 1.

FIG. 3 is a detail view of an example optical window consistent with embodiments.

FIG. 4 is an example cross-sectional view of an example optical window consistent with FIG. 3.

FIG. 5 is a perspective view of another example optical trocar consistent with embodiments.

FIG. 6 is a detail view of another example optical window consistent with some embodiments.

FIG. 7 is a perspective view of another example optical trocar consistent with embodiments.

FIG. 8 is an example flow chart consistent with embodiments disclosed herein.

The figures are rendered primarily for clarity and, as a result, are not necessarily drawn to scale. Moreover, various structure/components, including but not limited to fasteners, electrical components (wiring, cables, etc.), and the like, may be shown diagrammatically or removed from some or all of the views to better illustrate aspects of the depicted embodiments, or where inclusion of such structure/components is not necessary to an understanding of the various exemplary embodiments described herein. The lack of illustration/description of such structure/components in a particular figure is, however, not to be interpreted as limiting the scope of the various embodiments in any way.

### DETAILED DESCRIPTION

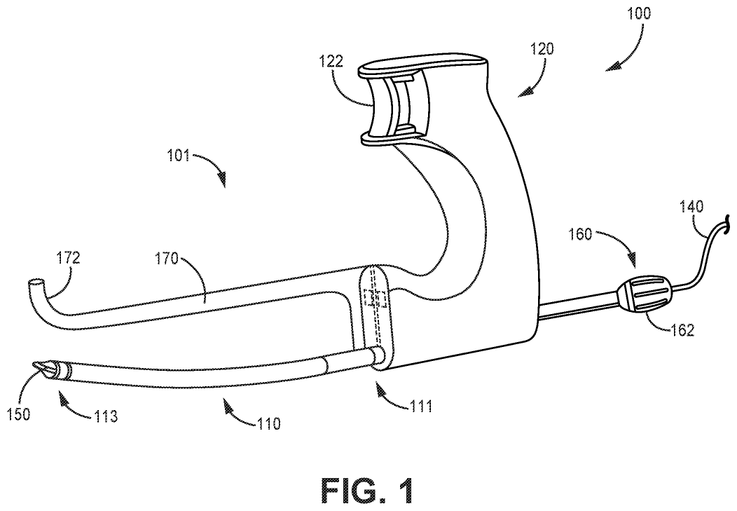

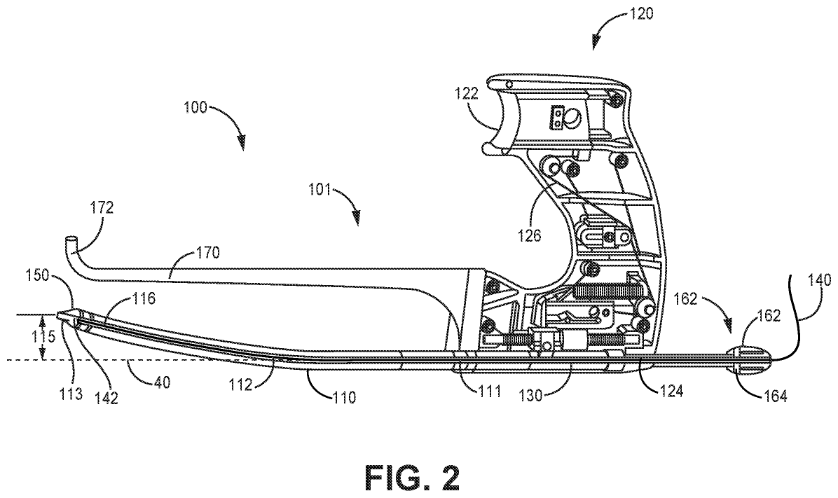

The technology disclosed herein generally relates to an optical trocar assembly, an example of which is depicted in FIGS. 1 and 2, where FIG. 2 is a cross-sectional view of the example optical trocar assembly 100 depicted in FIG. 1. The optical trocar assembly 100 is generally configured to penetrate the body tissue of a patient and provide visualization within the body of the patient during such penetration. The optical trocar assembly 100 generally has an optical trocar tool 101 and an endoscope 140. The optical trocar tool 101 has a handle 120, a tunneling shaft 110 coupled to the handle, and an optical window 44 on a distal end of the tunneling shaft 32.

The tunneling shaft 110 is generally configured to be inserted into and translated through the body tissue of a patient. The tunneling shaft 110 has a first end 111, which can be considered a proximal end 111, and a second or distal end 113. The tunneling shaft 110 is coupled to the handle 120 at the first end 111. The tunneling shaft 110 extends from the handle 120 to the distal end 113.

In various embodiments at least a portion of the tunneling shaft 110 extends in a curved orientation between the handle 120 and the distal end 113. For example, the tunneling shaft 110 may define a curve relative to axis 40, where axis 40 is straight line. The curved orientation of tunneling shaft 110 results in offset 115 between distal end 38 and axis 40 shown in FIG. 2. Offset 115 may range from approximately 0.35 inches to approximately 1.25 inches, such as, approximately 0.720 inches, although other examples are contemplated. In some instances, the curvature of tunneling shaft 110 may maintain the path of the distal tip close to posterior side of sternum and away from vital organs like lung or heart during a tunneling procedure.

In some examples, tunneling shaft 110 is curved about the entire length from proximal end 111 to distal end 113 or may include one or more sections that are substantially straight with one or more other sections that are curved. For example, a proximate portion of the tunneling shaft 110 extending directly from handle 120 may be approximately straight for some of the length of tunneling shaft 110 and then transition to a more distal portion of tunneling shaft 110 that is curved. In some examples, the curved portion of tunneling shaft exhibits a radius of curvature of about 15 inches to about 40 inches. In various embodiments the tunneling shaft 110 is bendable. As such, the curve of the tunneling shaft 110 can be adjusted by a user. Such a configuration may advantageously allow a user to modify the curvature of the tunneling shaft 110 to accommodate the anatomy of a particular patient and/or operating environment within which the tunneling shaft 110 is being used.

The tunneling shaft 110 can be constructed of a variety of types of material and combinations of materials. The tunneling shaft 110 can be constructed of metals (stainless steel, coated steel, titanium alloys, aluminum alloys and others) and plastics. Suitable plastic materials include but are not limited to acetal copolymer, polytetrafluoroethylene (PTFE) (e.g., TEFLON), polyether ether ketone (PEEK), polyphenylsulfone (PPSU) (e.g., RADEL), and polycarbonate. In some examples, the tunneling shaft 110 may be constructed of a material that allows for all or at least a portion of tunneling shaft 110 to be transparent along the length of shaft 110. The tunneling shaft 110 may be substantially rigid so a user can accurately control the position of the tip in relation to vital organs under visualization afforded by fluoroscopy or other techniques. In some examples, the distal end 113 of the tunneling shaft 110 has at least some metal components (like a metal blade) which will allow visualization using suitable medical imaging technology.

In some examples, the bendability and rigidity of the tunneling shaft 110 may be described in the context of possible forces that may act of shaft 110, e.g., before or during an implant procedure. In various embodiments, the tunneling shaft 110 is sufficiently rigid to maintain its shape during a tunneling operation where the tunneling shaft 110 is being inserted into and through the tissue of a patient. However, in many such embodiments, the tunneling shaft 110 is bendable by a user's hands absent the use of additional tools. In many such embodiments, the tunneling shaft 110 is configured to bend without collapse of the inner lumen 112. In some examples, tunneling shaft 110 exhibits substantially no flex when greater than about 5 pounds of force is applied to the distal end 113 from a direction perpendicular to the linear extension of the distal end 113. In some examples, tunneling shaft 110 does not “jam up” (e.g. can still deploy a cutting tool and/or allow for visualization via an optical window) when greater than about 5 pounds of force is applied to the distal end 113 from a direction perpendicular to the linear extension of the distal end 113.

The tunneling shaft 110 may exhibit any suitable shape and dimensions. In some embodiments the tunneling shaft 110 has a substantially circular cross-section perpendicular to the extension of the tunneling shaft 110, however other example cross-section shapes are contemplated. In some examples, the tunneling shaft 110 has an oval cross-section. The maximum cross-dimension (such as a diameter or diagonal dimension) of the tunneling shaft 110 can range from about 3 millimeters (mm) to about 15 mm, although other examples are contemplated. The length of tunneling shaft 110 from the proximal end 111 directly adjacent the handle 120 to the distal end 113 may range from about 4 inches to about 12 inches, although other examples are contemplated.

In various embodiments, the tunneling shaft 110 is tubular. The tunneling shaft 110 has an inner lumen 112 that extends from the proximal end 111 towards the distal end 113. The inner lumen 112 is a portion of an insertion pathway 130 that extends through the handle 120 and the tunneling shaft 110 to the optical window 150. The optical window 150 extends across the inner lumen 112 to seal the inner lumen 112 from the external environment on the distal end 113 of the tunneling shaft 110.

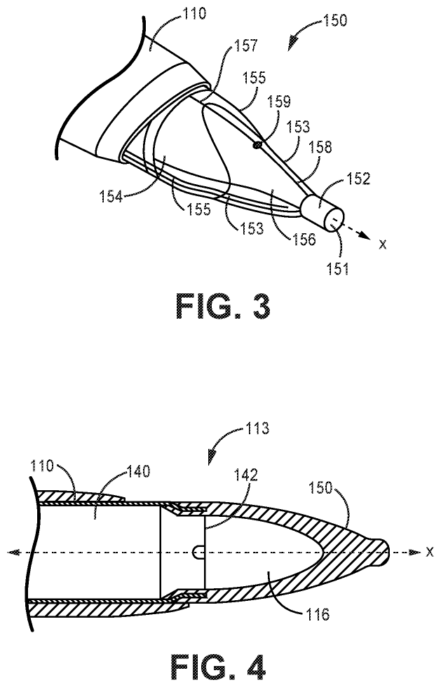

The distal end 113 of the tunneling shaft 110 has an optical window 150 that is constructed of a transparent or at least semi-transparent material to facilitate light ray transmission. FIG. 3 is a detail view of the distal end 113 of the tunneling shaft 110 and the optical window 150 consistent with some such embodiments. The optical window 150 can be formed of a transparent material, for example glass, quartz or clear plastics like polycarbonate (e.g., LEXAN) or acrylic. In some embodiments the tunneling shaft 110 is overmolded to the optical window 150, and in other embodiments the optical window 150 is overmolded to the distal end 113 of the tunneling shaft 110.

In some embodiments, the optical window 150 is shaped to facilitate blunt dissection when tunneled through tissue of patient. In some other embodiments, including the one depicted, the optical window 150 is configured to dissect between tissue planes without cutting or incising tissue. In some such embodiments the optical window 150 is considered to have a ‘dolphin-nose” or “parabolic” type shape. Other shapes are certainly contemplated. More particularly, the optical window 150 has a leading face that is an atraumatic guiding nub 152, a proximal section 154, and a central section 156. The optical window 150 generally has a hollow interior. The proximal section 154 has a pair of diametrically opposed convex surfaces 155. The central section 156 includes a pair of diametrically opposed concave surfaces 153. The atraumatic guiding nub 152 extends distally from the central section 156. The atraumatic guiding nub 152 is a cylindrical protrusion that has a rounded distal end 151. The rounded distal end 151 defines a radius of curvature dimensioned to be atraumatic to tissue. The atraumatic guiding nub 152 can generally have a circular profile in a direction perpendicular to the longitudinal extension of the optical window 150. The central section 156 can have an ovular profile in a direction perpendicular to the longitudinal extension of the optical window 150. The proximal section 154 can have a circular profile in a direction perpendicular to the longitudinal extension of the optical window 150.

The proximal section 154 of the optical window 150 further includes a pair of diametrically opposed outer surfaces 157 which are generally linear and/or convex (where only one outer surface 157 is visible in FIG. 3, which is a “top” surface relative to FIG. 3). The central section 156 also includes a pair of opposed outer surfaces 158 which are convex (where only one outer surface 157 is visible in FIG. 3, which is a “top” surface relative to FIG. 3). Thus, the central section 156 of the optical window 150 is inclusive of both generally concave surfaces 153 and generally convex surfaces 158 that are circumferentially spaced around the optical window 150.

The atraumatic guiding nub 152 permits initial insertion within a pre-formed opening, e.g., a pre-cut scalpel incision, in the tissue and facilitates the advancement of the optical window 150 between the tissue layers to gently dissect tissue, without any cutting or incising of the tissue. After initial insertion and continued distal insertion, the central section 156 and the proximal section 154 continue to gently enlarge the opening in tissue by further dissecting the tissue planes, e.g. by the rounded outer surfaces of optical window separating the tissue planes during a clocking, twisting, or rocking motion thereof.

Referring back to FIGS. 1-2, in various embodiments the endoscope 140 is configured to be inserted in the handle 120 and advanced towards the optical window 150 along the insertion pathway 130. FIG. 4 depicts a detail cross-sectional view of the endoscope 140 inserted towards the distal end 113 of the tunneling shaft 110. An “endoscope” is defined herein as an instrument that enables visualization of the internal environment within the body of a patient. The endoscope 140 can be inserted into the insertion pathway 130 via a proximal opening 124 in handle 120. The endoscope 140 can have a distal end 142 that includes a visualization component configured to be positioned adjacently to the optical window 150. The visualization component can include an illumination device (such as a light), a camera, and/or other visualization devices and combinations of visualization devices. The distal end 142 of the endoscope 140 can be advanced through the insertion pathway 130 to the distal end 113 of the tunneling shaft 110 to a position adjacent the optical window 150. In this manner, a surgeon or other user may visualize the environment surrounding the distal end 113 when the tunneling shaft 110 is advanced through the tissue of a patient during a medical procedure.

In some embodiments, an air gap is formed between the distal end 142 of the endoscope 140 and the optical window 150. In some embodiments, an optical material 116 can be disposed in the insertion pathway 130 between the endoscope 140 and the optical window 150. The optical material 116 can generally be configured to facilitate visualization of the surrounding environment by the endoscope 140 through the optical window 150. The optical material 116 may advantageously eliminate glare in some implementations. In some implementations, the optical material 116 may advantageously mitigate condensation on the optical window 150. In some implementations, the optical material 116 is an optically clear substance that facilitates transmission of optical signals. In various embodiments the optical material 116 is in contact with the optical window 150. The optical material 116 can be a liquid, but in other embodiments the optical material can be a solid. In some embodiments the optical material 116 is a saline solution. In some embodiments the optical material 116 is water. In some embodiments the optical material 116 is an optical silicone. In various embodiments the optical material 116 is a compressible material that allows linear translation of the endoscope 140 along the insertion pathway 130. In some embodiments the optical material 116 extends along most of the length of the insertion pathway 130. In some embodiments the optical material 116 is disposed towards a portion of the insertion pathway 130 such as towards the distal end 113 of the tunneling shaft 110.

In some embodiments the assembly consistent with the technology disclosed herein has an orientation indicator 159 disposed on the optical window, an example of which is depicted in FIG. 3. The orientation indicator 159 is a visual marker that is generally configured to orient a user to the environment outside of the optical window 150 that is visible through the endoscope 140. In various embodiments, the orientation indicator 159 is asymmetric relative to a longitudinal axis x of the optical window 150. The orientation indicator 159 can be an area of ink or other colorant on the optical window 150 that is visible to a user. In some other embodiments the orientation indicator 159 is a visual aberration on the optical window 150 formed through melting a small area on the optical window 150 and/or depositing a visible material on the optical window 150.

Returning to FIG. 2, the optical trocar assembly 100 consistent with the technology disclosed herein generally has a constriction mechanism 160 coupled to the handle 120. The constriction mechanism 160 defines a portion of the insertion pathway 130. The constriction mechanism 160 is configured to selectively constrict the insertion pathway 130, particularly around at least a portion of the endoscope 140. The constriction mechanism 160 is configured to selectively fix the endoscope 140 to the tunneling shaft 110 and may advantageously prevent shifting of the endoscope 140 relative to the tunneling shaft 110 when the tunneling shaft 110 is advanced through the tissue of a patient.

In various embodiments, the constriction mechanism 160 is configured to selectively frictionally engage at least a portion of a perimeter region of the endoscope 140 within the insertion pathway 130. In various embodiments the constriction mechanism 160 is configured to be selectively disengaged from the endoscope and then selectively reengaged such that the endoscope can be repositioned relative to the insertion pathway 130. The constriction mechanism 160 can be adjustable in a variety of embodiments to selectively frictionally engage endoscopes having varying cross-sectional cross-dimensions (such as a diameter). In some embodiments, the magnitude of the constriction (such as the pressure) of the constriction mechanism 160 is adjustable by a user. In various embodiments, the constriction mechanism 160 has a pressure limiter that is configured to limit the pressure exerted on the endoscope 140 by the constriction mechanism 160. The pressure limiter can be configured to limit constriction of the insertion pathway 130. Such a configuration may advantageously avoid over compression of the endoscope's electrical wiring.

In various embodiments, the constriction mechanism 160 has a manually engageable knob 162. The manually engageable knob 162 is configured to be rotated by a user around a knob central axis to selectively constrict or expand the portion of the insertion pathway 130 defined by the constriction mechanism 160. In some embodiments the manually engageable knob 162 is in operative communication with radial protrusions that are configured to advance radially into the insertion pathway 130 when the manually engageable knob 162 is rotated in one direction and the radial protrusions are configured to advance radially outward from the insertion pathway 130 when the manually engageable knob 162 is rotated in an opposite direction. In another embodiment the manually engageable knob 162 can be a rotatable pin such as a pin vice or set screw that is advanced into the insertion pathway 130 to fix the endoscope 140 relative to the insertion pathway 130 and is advanced outward from the insertion pathway 130 to unfix the endoscope 140 relative to the insertion pathway 130.

In the current example, the constriction mechanism 160 has an inner diaphragm 164 that defines a portion of the insertion pathway 130. Upon rotation of the manually engageable knob in a first direction, the diaphragm 164 advances radially inward towards the endoscope 140. Upon rotation of the manually engageable knob 162 in a second direction, the diaphragm 164 advances radially outward from the endoscope 140.

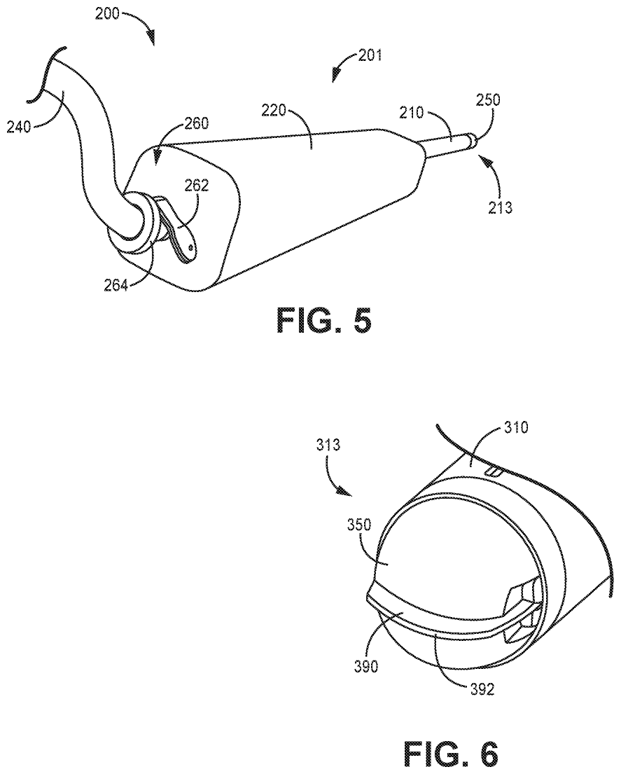

While in the current example the constriction mechanism 160 has a manually engageable knob 162, in some other embodiments the constriction mechanism 160 has a lever that is pivotably coupled to the optical trocar in communication with the insertion pathway. FIG. 5 is a perspective view of another example optical trocar assembly 200 consistent with some embodiments. The optical trocar assembly 200 generally has an endoscope 240 and an optical trocar tool 201 having a handle 220, a tunneling shaft 210 coupled to the handle 220, an optical window disposed 250 at a distal end 213 of the tunneling shaft and an insertion pathway extending through the handle and the tunneling shaft to the optical window, which is not currently visible, but is similar to the insertion pathway discussed above and shown in FIG. 2. The endoscope 240 is configured to be inserted in the handle 220 to the optical window 250 along the insertion pathway. A constriction mechanism 260 is coupled to the handle 220, which defines a portion of the insertion pathway. The constriction mechanism 260 is configured to selectively constrict the insertion pathway.

The components depicted in the current figure are generally consistent with the descriptions of components discussed above with reference to FIGS. 1-4, except where such descriptions conflict with FIG. 5 or the current description. Unlike previous embodiments, in the current example the constriction mechanism 260 has a manually engageable lever 262. The lever 262 is pivotably coupled to the handle 220. The manually engageable lever 262 is compressibly disposed around the proximal end of the insertion pathway, which is defined by a pathway ring 264 coupled to the proximal end of the handle 220. The pathway ring 264 can be a compressible and/or flexible material such as rubber or another elastomeric material. When the lever 262 is pivoted to an engaged position, the pathway ring 264 constricts around outer perimetric boundary of the endoscope 240 to frictionally engage the outer perimetric boundary of the endoscope 240. When the lever 262 is pivoted to a disengaged position, the pathway ring 264 expands to reduce or eliminate its frictional engagement of the outer perimetric boundary of the endoscope 240.

It is noted that the example optical trocar assembly 100 of FIGS. 1 and 2 has a guide member 170 extending from the handle 120. The guide member 170 is adjacent to and coplanar with the tunneling shaft 110. The tunneling shaft 110 is curved towards guide member 88. The guide member 170 may help a surgeon or other operator in advancing the tunneling shaft 110 after the distal end 113 is inserted into a patient. In some examples, curved distal end 172 of the guide member 170 may be configured to ‘ride’ across the skin without binding on the skin during such a procedure. In this manner, the guide member 170 may limit the depth below the skin of the tunneling shaft 110 during the procedure. Further, the curvature of tunneling shaft 110 toward the guide member 170 can cause the distal end 113 to ‘ride’ adjacent an inside surface of the patient's sternum, as an example, during the advancement thereof as an additional aid to the operator. In some examples, the distance between the guide member 170 and the tunneling shaft 110 can be adjusted as desired by a surgeon or other operator, e.g., based on the physical characteristics of a patient. It is noted that in other implementations, such as the implementation depicted in FIG. 5, a guide member can be omitted.

In the example optical trocar assembly 100 of FIGS. 1-2, the handle 120 has a trigger 122. The trigger 122 can be operatively coupled to tool components via a mechanical communication chain 126 (FIG. 2) for engaging particular features. In some embodiments the trigger 122 can be operatively coupled to the endoscope 140 to selectively engage endoscope features such as lighting or recording features. In some other embodiments the trigger 122 is operatively coupled to a cutting tool, an example of which is described below with reference to FIG. 6. It is noted that in the example depicted in FIG. 5, the handle 220 does not have a trigger.

FIG. 6 depicts a detail view of another example distal end 313 of a tunneling shaft 310 consistent with some embodiments. The distal end 313 of the tunneling shaft 310 has an optical window 350 consistent with discussions above, and a deployable cutting tool 390 coupled to the optical window 350. Such an embodiments can be consistent with the examples of FIGS. 1-2 where the trigger 122 on the handle 120 is in operative communication with the cutting tool.

The cutting tool 390 can take the form of a knife blade, scalpel blade, or other tool with a sharp edge 392 that is configured to cut through tissue, such as, scar tissue, of patient while the tunneling shaft 310 is tunneled in the tissue of a patient to a target location. The cutting tool 390 can be configured to be selectively actuated by a surgeon or other user from a recessed position (not currently depicted) to a deployed position (as shown in FIG. 6). When the cutting tool 390 is in a recessed position, the lead or cutting edge of cutting tool 390 is recessed into the distal end 313 of the tunneling shaft 310 such that the outer surface of optical window 350 is coextensive with the leading edge of the sharp edge 392, allowing for blunt dissection of tissue while the tunneling shaft 310 is advanced through patient tissue. Conversely, when the cutting tool 390 is in the deployed position, the cutting tool 390 defines the leading edge of the tunneling shaft 310, allowing for tissue, such as scar tissue, to be cut by the tool by sharp dissection rather than blunt dissection.

Any suitable mechanism may be used to transition the cutting tool 390 between a recessed position and deployed position (FIG. 6). The cutting tool 390 can be mechanically coupled to handle 120 through the tunneling shaft 310 such that the actuation of trigger 122 (FIGS. 1-2) translates the cutting tool 390 outward in the longitudinal direction beyond the optical window 350 to define the leading face on the distal end 313 of the tunneling shaft 310. In such a deployed position, the sharp/leading edge 392 of the cutting tool 390 may extend about 0.25 mm to about 2 mm beyond the distal end 313 of the tunneling shaft 310. Put another way, in the deployed position, the sharp/leading edge 392 of the cutting tool 390 may extend about 0.25 mm to about 2 mm beyond the leading edge of the distal end 313 (for example, the outer surface of the optical window 350) when the cutting tool 390 is in the recessed position.

In some examples, the engagement (such the depressing) the trigger (122, FIG. 2) actuates the cutting tool 390 from the recessed position to the deployed position and the cutting tool 390 can remain in the deployed position until the trigger is released. Alternatively, the tunneling shaft 310 can be configured such that the actuation of trigger (for example, depression or depression and release of the trigger) may result in the cutting tool 390 being deployed from the recessed position to the deployed position and then automatically returned to the recessed position, for example, after the cutting tool 390 advances forward a pre-set distance. In some examples, a surgeon or other user may hold the handle (120, FIG. 1) of the tool stationary when the trigger is depressed to control the length of tissue that is dissected by the cutting tool 390, which approximately corresponds to the length at which the sharp/leading edge 392 of the cutting tool 390 extends out of the distal end 313 when the trigger is depressed to move the cutting tool 390 into the deployed position. Alternatively, or additionally, a surgeon or other user may advance the tunneling shaft 310 forward by the handle when cutting tool 390 is held in the deployed position, where the length of tissue dissection by cutting tool 390 corresponds generally to the length that tunneling shaft 310 is advanced under the control of the surgeon or other user.

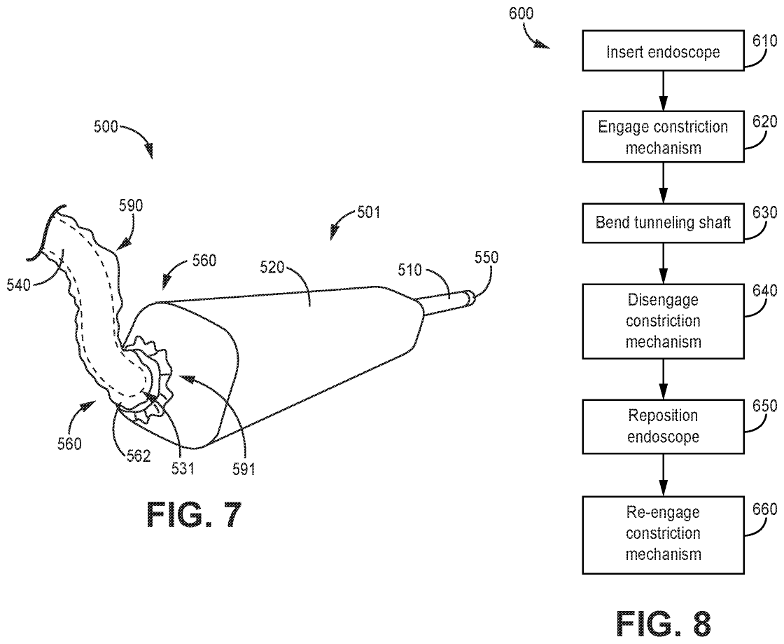

Technology consistent with the present disclosure may advantageously allow re-use of an endoscope with different optical trocar tools. In some embodiments, the optical trocar tools are generally configured for a single-use and are disposable. In some implementations, the endoscopes are configured for multiple uses and are generally not considered disposable after a single use. Referring to FIG. 7, which is yet another example optical trocar assembly 500 consistent with embodiments generally discussed above, the tunneling shaft 510, the optical window 550 and the handle 520 can generally be hermetically sealed to isolate a distal portion (not shown) of the endoscope 540 from the outside environment including the tissue and fluids of a patient during a medical procedure. In some further embodiments, the optical trocar assembly 500 of the current technology incorporates an isolation sheath 590 that is configured to isolate and protect a portion of the endoscope 540 extending beyond the proximal end of the handle 520 in the environment outside of the optical trocar tunneling shaft 510 and handle 520.

The isolation sheath 590 is generally a tubular structure that is configured to receive a portion of the length of the endoscope 540 extending beyond the proximal end of the handle 520. The isolation sheath 590 generally forms an outer circumferential barrier between the outside environment and a length of an outer surface of the endoscope 540 extending beyond the optical trocar. In some embodiments the isolation sheath 590 is a nonbreathable material. In some embodiments the isolation sheath 590 obstructs liquid flow therethrough. In some embodiments the isolation sheath 590 is configured to be sterile. The isolation sheath 590 can be constructed of a polymeric material such as polyethylene.

The isolation sheath 590 can have a first end 591 that is configured to be sealed about a proximal end 531 of the insertion pathway (not currently visible but shown as element 130 in FIG. 2). In some embodiments the first end 591 of the isolation sheath 590 is configured to be sealed to the handle 520. The first end 591 of the isolation sheath 590 can be sealed such as by welding, such as heat welded or ultrasonic welded, adhered with an adhesive, compressed, or otherwise sealed about the proximal end 531 of the insertion pathway. In some embodiments, the first end 591 of the isolation sheath 590 is sealed to the handle 520. In some other embodiments, the first end 591 of the isolation sheath 590 is sealed to a pathway ring around the insertion pathway, such as the pathway ring 264 shown in FIG. 5 and described above. In some embodiments the first end 591 of the isolation sheath 590 is sealably fixed to an intermediate component such as a bayonet connector or snap fit connector that is sealably coupled to the handle 520 or other component defining the proximal end of the insertion pathway (130) relative to the optical trocar tool 501.

In the current example, the constriction mechanism 560 includes a manually engageable knob 562 rotatably coupled to the proximal end of the handle 520. The manually engageable knob 562 is a ring defining a central opening that defines the proximal end 531 of the insertion pathway. The central opening of the manually engageable knob 562 is configured to receive the endoscope 540 and frictionally engage the outer surface of the endoscope 540 upon engagement such as by manually rotating the knob 562 around its central opening. In this example, the first end 591 of the isolation sheath 590 is sealed to the constriction mechanism 560, such as the manually engageable knob 562, around the proximal end 531 of the insertion pathway.

The isolation sheath 590 can be constructed of a thin, flexible material that is configured to be manually extended along the length of the endoscope 540 from the handle 520. Prior to engagement, the isolation sheath 590 can have a first, un-extended length. Upon insertion of the endoscope into the proximal end 531 of the insertion pathway and in preparation for or during a procedure, the isolation sheath 590 can be manually extended along the length of the endoscope 540 to prevent contamination of the length of the endoscope 540 beyond the handle. The isolation sheath 590 can have an extended length that is greater than the un-extended length.

FIG. 8 depicted a flow chart consistent with some methods in accordance with the technology disclosed herein. The method 600 can be consistent with performing a procedure on a patient. An endoscope is inserted 610, a constriction mechanism is engaged 620, a tunneling shaft is bent 630, the constriction mechanism is disengaged 640, the endoscope is repositioned 650, and the constriction mechanism is reengaged 660. The features and configurations of the various components discussed herein can be consistent with the descriptions of corresponding components found above.

The endoscope is generally inserted 610 through an insertion pathway of a trocar assembly. The endoscope is inserted 610 from a proximal end to an optical window disposed on a distal end of the trocar assembly. In some embodiments where optical fluid is disposed in the insertion pathway, inserting the endoscope 610 includes translating the endoscope through the optical material disposed in the insertion pathway. In some embodiments, optical fluid is disposed in the insertion pathway as part of the method described herein. The optical fluid can be disposed in the insertion pathway prior to insertion of the endoscope through the insertion pathway. In some other embodiments, however, the optical fluid can be disposed in the insertion pathway after insertion of the endoscope through the insertion pathway 610.

The constriction mechanism is engaged 620 to secure the endoscope relative to the insertion pathway. The constriction mechanism is generally positioned towards the proximal end of the trocar assembly. In some embodiments where the constriction mechanism has a lever, engaging the constriction mechanism includes pivoting the lever to an engaged position. In some embodiments where the constriction mechanism has a rotatable knob, engaging the constriction mechanism includes turning the knob to an engaged position. In some embodiments the magnitude of the constriction can be manually adjusted. In some such embodiments where an inner diaphragm forms a portion of the constriction mechanism, adjusting the magnitude of the constriction includes adjusting the inner diaphragm. In various embodiments the constriction mechanism is configured to limit pressure on the endoscope.

Bending the tunneling shaft 630 of the trocar assembly can include, for example, manually grasping the tunneling shaft 630 by a user and applying a bending force to the tunneling shaft that is sufficient to overcome the stiffness of the tunneling shaft 630. In some embodiments, the tunneling shaft is bent 630 to accommodate the present operating environment, such as the anatomy of the particular patient on which the procedure is performed.

In various embodiments, bending the tunneling shaft 630 while the constriction mechanism is engaged will translate the distal end of the endoscope relative to the optical window due to the differences in the radius of curvature forming the bend between the tunneling shaft and the endoscope. As such, before or after bending the tunneling shaft 630, the constriction mechanism can be disengaged 640 to unfix the endoscope relative to the insertion pathway. The endoscope can then be repositioned within the insertion pathway 650 such as by manually translating the endoscope along the insertion pathway so that the distal end of the endoscope is positioned desirably relative to the optical window. When the desired position to attained, the constriction mechanism can be re-engaged to again secure the endoscope relative to the insertion pathway 660.

In various embodiments, the procedure can include inserting the distal end of the trocar assembly through an incision in a patient. Any one of the steps discussed above can occur before, after, or during insertion of the distal end of the trocar assembly through the incision in the patient. In some embodiments that have been discussed above, the optical window has an orientation indicator. In such embodiments the user can consult the orientation indicator, such as during insertion of assembly through the incision in the patient to orient the device in a desirable orientation. In come embodiments where the optical trocar assembly incorporates a deployable cutting blade coupled to the distal end of the optical trocar, the cutting blades can be deployed by the user.

It should also be noted that, as used in this specification and the appended claims, the phrase “configured” describes a system, apparatus, or other structure that is constructed to perform a particular task or adopt a particular configuration. The word “configured” can be used interchangeably with similar words such as “arranged”, “constructed”, “manufactured”, and the like.

All publications and patent applications in this specification are indicative of the level of ordinary skill in the art to which this technology pertains. All publications and patent applications are herein incorporated by reference to the same extent as if each individual publication or patent application was specifically and individually indicated by reference. In the event that any inconsistency exists between the disclosure of the present application and the disclosure(s) of any document incorporated herein by reference, the disclosure of the present application shall govern.

This application is intended to cover adaptations or variations of the present subject matter. It is to be understood that the above description is intended to be illustrative, and not restrictive, and the claims are not limited to the illustrative embodiments as set forth herein.

The following examples are a non-limiting list of clauses in accordance with one or more techniques of this disclosure.

Example 1. An assembly comprising: a handle; a tunneling shaft coupled to the handle, the tunneling shaft extending from the handle to a distal end, wherein at least a portion of the tunneling shaft extends in a curved orientation between the handle and the distal end; an optical window disposed at the distal end of the tunneling shaft; an insertion pathway extending through the handle and the tunneling shaft to the optical window; a constriction mechanism coupled to the handle defining a portion of the insertion pathway, wherein the constriction mechanism is configured to selectively constrict the insertion pathway; and an endoscope configured to be inserted in the handle to the optical window along the insertion pathway.

Example 2. The assembly of Example 1, wherein the constriction mechanism comprises an inner diaphragm defining the portion of the insertion pathway.

Example 3. The assembly of any one of Examples 1 or 2, wherein the constriction mechanism comprises a manually engageable knob.

Example 4. The assembly of any one of Examples 1-3, wherein the constriction mechanism is configured for selective disengagement and reengagement of the endoscope.

Example 5. The assembly of any one of Examples 1-4, further comprising optical material disposed in the insertion pathway, wherein the optical material is in contact with the optical window.

Example 6. The assembly of any one of Examples 1-5, further comprising an orientation indicator on the optical window.

Example 7. The assembly of any one of Examples 1-6, wherein the constriction mechanism has a pressure limiter that is configured to limit constriction of the insertion pathway.

Example 8. The assembly of any one of Examples 1-7, wherein the magnitude of constriction of the constriction mechanism is adjustable by a user.

Example 9. The assembly of any one of Examples 1-8, wherein the tunneling shaft is bendable.

Example 10. A method comprising: inserting an endoscope through an insertion pathway of a trocar assembly from a proximal end to an optical window disposed on a distal end of the trocar assembly; engaging a constriction mechanism that is positioned towards the proximal end of the trocar assembly to secure the endoscope relative to the insertion pathway; bending a tunneling shaft of the trocar assembly; disengaging the constriction mechanism; repositioning the endoscope within the insertion pathway; and re-engaging the constriction mechanism to secure the endoscope relative to the insertion pathway.

Example 11. The method of Example 10, wherein engaging the constriction mechanism comprises pivoting a lever.

Example 12. The method of any one of Examples 10 or 11, wherein engaging the constriction mechanism comprises turning a knob.

Example 13. The method of any one of Examples 1-12, further comprising disposing optical material within the insertion pathway.

Example 14. The method of any one of Examples 1-13, wherein inserting the endoscope further comprises translating the endoscope through an optical material disposed in the insertion pathway.

Example 15. The method of any one of Examples 1-14, wherein the constriction mechanism is configured to limit pressure on the endoscope.

Example 16. The method of any one of Examples 1-15, wherein the optical window comprises an orientation indicator.

Example 17. The method of any one of Examples 1-16, further comprising adjusting the magnitude of the constriction.

Example 18. The method of example 17, wherein adjusting the magnitude of the constriction comprises adjusting an inner diaphragm.

Example 19. The method of any one of Examples 1-18, further comprising inserting the distal end through an incision in a patient.

Example 20. The method of any one of Examples 1-19, further comprising deploying cutting blades coupled to the distal end.

1. An assembly comprising:
a handle;
a tunneling shaft coupled to the handle, the tunneling shaft extending from the handle to a distal end, wherein at least a portion of the tunneling shaft extends in a curved orientation between the handle and the distal end;
an optical window disposed at the distal end of the tunneling shaft;
an insertion pathway extending through the handle and the tunneling shaft to the optical window;
a constriction mechanism coupled to the handle defining a portion of the insertion pathway, wherein the constriction mechanism is configured to selectively constrict the insertion pathway; and
an endoscope configured to be inserted in the handle to the optical window along the insertion pathway.

2. The assembly of claim 1, wherein the constriction mechanism comprises an inner diaphragm defining the portion of the insertion pathway.

3. The assembly of claim 1, wherein the constriction mechanism comprises a manually engageable knob.

4. The assembly of claim 1, wherein the constriction mechanism is configured for selective disengagement and reengagement of the endoscope.

5. The assembly of claim 1, further comprising optical material disposed in the insertion pathway, wherein the optical material is in contact with the optical window.

6. The assembly of claim 1, further comprising an orientation indicator on the optical window.

7. The assembly of claim 1, wherein the constriction mechanism has a pressure limiter that is configured to limit constriction of the insertion pathway.

8. The assembly of claim 1, wherein the magnitude of constriction of the constriction mechanism is adjustable by a user.

9. The assembly of claim 1, wherein the tunneling shaft is bendable.

10. The assembly of claim 2, wherein the constriction mechanism comprises a manually engageable knob.

11. The assembly of claim 2, wherein the constriction mechanism is configured for selective disengagement and reengagement of the endoscope.

12. The assembly of claim 2, further comprising optical material disposed in the insertion pathway, wherein the optical material is in contact with the optical window.

13. The assembly of claim 2, further comprising an orientation indicator on the optical window.

14. The assembly of claim 2, wherein the constriction mechanism has a pressure limiter that is configured to limit constriction of the insertion pathway.

15. The assembly of claim 2, wherein the magnitude of constriction of the constriction mechanism is adjustable by a user.

16. The assembly of claim 2, wherein the tunneling shaft is bendable.

17. The assembly of claim 3, wherein the constriction mechanism is configured for selective disengagement and reengagement of the endoscope.

18. The assembly of claim 3, further comprising optical material disposed in the insertion pathway, wherein the optical material is in contact with the optical window.

19. The assembly of claim 3, further comprising an orientation indicator on the optical window.

20. The assembly of claim 3, wherein the constriction mechanism has a pressure limiter that is configured to limit constriction of the insertion pathway.
